(19) United States
(12) Patent Application Publication
(10) Pub. No.: US 2024/0074962 A1
(43) Pub. Date: Mar. 7, 2024

(54) Hair Care Product and Kit

(71) Applicant: Ying Chun Liu, Guelph (CA)

(72) Inventor: Ying Chun Liu, Guelph (CA)

(21) Appl. No.: 18/463,029

(22) Filed: Sep. 7, 2023

Related U.S. Application Data
(60) Provisional application No. 63/374,817, filed on Sep. 7, 2022.

(51) Int. Cl. A61K 8/73; A61K 8/02; A61K 8/34; A61K 8/365; A61K 8/44; A61Q 5/06

(52) U.S. Cl. CPC A61K 8/732; A61K 8/0241; A61K 8/345; A61K 8/365; A61K 8/44; A61Q 5/06; A61K 2800/95

(57) ABSTRACT

A dry formulation useful to prepare a hair care product is provided. The formulation comprises: i) a water soluble film-forming polymer; ii) at least one hair product ingredient, and iii) optionally, a pH adjuster in an amount sufficient to establish a pH in the range of about 3.0 to about 7.5. The dry formulation may be further processed, for example, prepared into tablets or other dosage forms, for ease of use. Methods of making the hair care product are also provided, as well as kits containing the formulation.

### FIELD OF THE INVENTION

The present invention generally relates to a hair care product, and in particular, to a dry formulation useful to prepare a hair care product. The present invention also relates to a kit comprising the formulation and a container.

### BACKGROUND OF THE INVENTION

There are a large variety of fluid hair products that contain polymers including: hair spray, hair mousse, hair gel, wax, pomade, volumizer, shampoo, conditioner, oils and serums including anti-frizz serums, heat protectant spray and curl enhancing cream to name a few.

Over the course of the past few decades, fluid hair products, and specifically hair care products, have become increasingly reliant on polymers. While the original uses of polymers in hair products were as fixative agents and viscosity controlling additives, more recently, new polymers have been used for conditioning and style control.

Hair care products are conventionally purchased by consumers in pre-packaged one-time use containers for later disposal. The product inside the pre-packaged one-time use container is typically in a liquid or gel form and once exhausted, the container is discarded and a completely new one-time use container with product must be purchased.

It would be advantageous to provide an alternative to the costly and environmentally harmful packing regime that is currently dominating the market. There is currently no consumer prepared alternative to the pre-packaged hair care products provided in one-time use containers on the market.

Tablet formation is a well-established art. Generally, the process involves preparing a formulation containing an active ingredient, excipient and fillers to aid in the flow and stabilization of the formulation to be compressed or tableted. Excipients are substances that are included in a pharmaceutical dosage form not for their direct therapeutic action, but to aid the manufacturing process, and to protect, support and/or enhance stability. Fillers, often referred to as diluents, are inert ingredients that can significantly affect the chemical and physical properties of the final tablet.

When forming a tablet, the formulation may be blended into a product for direct compression or go through a process of wet or dry granulation. In the direct compression method, formulation ingredients are mixed in a blender and can be processed and compressed into tablets without any ingredients having to be changed. Dry granulation uses mechanical compression or compaction to facilitate the agglomeration of dry powder particles, while the wet granulation uses granulation liquid (binder/solvent) to facilitate the agglomeration by formation of wet mass by adhesion.

Care products in single use dosage forms have been made previously. For example, CA 2456535 describes personal care compositions comprising an oral, dental or skin hygiene composition admixed with an agar-based gelling agent to yield homogenous, semi-solid single-use lumps or beads. The single-use lumps or beads are mixed with hot water to release the desired oral, dental or skin care product.

U.S. Pat. No. 9,316,580 describes a system for performing a customized treatment of keratinous fibers. The system comprises an optical reader, a computational unit for predicting an outcome of the treatment; and a mixing unit to receive active agents, at least one of which is in tablet form. The described tablets comprise a water-insoluble super-disintegrant and at least one colour-imparting agent. The patent describes disintegrating at least one of the tablets in an aqueous medium to obtain a composition useful to treat keratinous fibers.

However, the prior art does not describe a dry formulation comprising water soluble film-forming polymers with or without at least one hair ingredient useful to create a desired hair product.

In view of the foregoing, it would be desirable to provide a hair care product that overcomes at least one disadvantage of prior hair care products.

### BRIEF DESCRIPTION OF THE INVENTION

A formulation useful to prepare a hair care product has now been developed that is provided in dry form. The formulation is beneficial for travel and provides an environmentally friendly alternative to pre-packaged hair care products.

In one aspect, a dry formulation useful to prepare a hair care product is provided. The powder comprises: i) a water soluble film-forming polymer; ii) at least one hair product ingredient; and optionally iii) a pH adjuster in an amount sufficient to establish a pH in the range of about 3.0 to about 7.5.

In another aspect, a kit is provided comprising: i) an amount of a dry formulation useful to prepare a hair care product as defined above; and ii) a container.

In another aspect, a hair product tablet is provided comprising: i) a water soluble film-forming polymer; ii) a tablet-binding agent and/or diluent; iii) a lubricant; and iv) at least one hair product ingredient.

In another aspect of the invention, a kit is provided comprising: i) one or more tablets comprising a water soluble film-forming polymer, a binding agent and/or a diluent, a lubricant, and at least one hair product ingredient; and ii) a container.

In another aspect of the invention, a kit is provided comprising: i) one or more tablets comprising a water soluble film-forming polymer, a binding agent and/or a diluent and a lubricant; and a container suitable for application of a hair care product.

In a further aspect of the invention, a method of making a hair care product is provided comprising the steps of: combining i) a dry formulation comprising a water soluble film-forming polymer; at least one hair product ingredient, and optionally, a pH adjuster in an amount sufficient to establish a pH in the range of about 3.0 to about 7.5; or ii) a tablet comprising a water soluble film-forming polymer, a binding agent and/or a diluent, a lubricant and at least one hair product ingredient; and iii) admixing the dry formulation or tablet with an amount of an aqueous solution sufficient to product the hair care product.

### DETAILED DESCRIPTION OF THE INVENTION

A dry formulation useful to prepare a hair care product is provided comprising: i) a water soluble film-forming polymer; ii) at least one hair product ingredient, and iii) optionally, a pH adjuster in an amount sufficient to establish a pH in the range of about 3.0 to about 7.5.

The term “hair care product” is used herein to refer to hair spray, hair mousse, hair gel, and other hair care products used for hair styling, frizz control and hair protection.

The present formulation comprises a water soluble film-forming polymer that is appropriate for use in a hair care product. The phrase “water soluble” as used herein is meant to encompass any film-forming polymer that dissolves in a given aqueous solution, including polymers that are soluble, very soluble and completely soluble. Examples of suitable water soluble film forming polymers include, but are not limited to: hydrolyzed corn starch, natural gum such as xanthan gum, tragacanth gum, guar gum, acacia gum, arabic gum and carrageenan gum, polysaccharides such as pullulan, amylose, alginate, carrageenan, pectin, dextrin, dextran, chitin, chitosan, levan, elsinan, scleroglucan and alternant, polyvinyl pyrrollidone polymer (PVP), carbopol, polyacrylamide, polyacrylic acid or a salt thereof such as sodium salt, acrylic-based polymers such as acrylate copolymers, styrene acrylate copolymer, hydroxyester acrylate copolymer and PVP acrylate copolymer, polyvinylpyrrolidone/vinyl acetate copolymer (VP/VA copolymer), vinyl acetate polymer, vinyl acrylate copolymer, polyurethane, acrylic polyurethane hydride polymers, polyvinyl alcohol, hydroxypropylmethyl cellulose, hydroxyethyl cellulose, hydroxypropyl cellulose, carboxymethyl cellulose, glycolide, polylactide, methyl methacrylate copolymer, carboxyvinyl polymer, polyquaternium polymers (e.g. polyquaternium-11, polyquaternium-4, polyquaternium-46, polyquaternium-16, polyquaternium-39, polyquaternium-2, polyquaternium-28), butyl or ethyl esters of polyvinyl methyl ether/maleic acid (PVM/MA) copolymers, and guar derivatives. The formulation may also include combinations of two or more of such water-soluble film-forming polymers.

The present formulation will generally comprise one or more water soluble film forming polymers in an amount in the range of about 5% to about 99.9% by wt. Preferably, the amount of film-forming polymer in the formulation is at least 10%, 20%, 30%, 40%, 50%, 60%, 70%, 80% or 90% by weight of the formulation, for example, in the range of 20-90% by wt, or 30-80% by wt. or 40-70% by wt.

The present formulation may also comprises a pH adjuster. A pH adjuster is a chemical used to alter the pH or potential hydrogen level. The potential hydrogen level is simply a measure of the activity of the hydrogen ion. Solutions with high levels of hydrogen ions are acidic and have a pH reading below 7. Alternatively, solutions with low levels of hydrogen ions have an increase in hydroxide ions, are alkaline and have a pH reading above 7.

Hair has a protective layer called the cuticle. The cuticle, when seen under a microscope, is made up of many scales that overlap. When in place and properly overlapping, they keep moisture in the hair shaft. Flat cuticle scales protect the hair more and give a smooth, shiny appearance to the hair. Alkaline products tend to lift up and open cuticle scales resulting in dryness and breakage. For this reason, it is important that the pH of hair products be maintained at or below about 7.5 on the pH scale. It is equally important to provide a product that is not too acidic on the pH scale. Ideally, the pH of the hair product will generally fall between about 3.0 and about 7.5 on the pH scale, for example, 5.0-6.0. Thus, a pH adjuster may be required if the pH of the formulation is not within the desired pH range. Examples of pH adjusters suitable for use in hair care products include, but are not limited to, lactic acid, glycolic acid, citric acid, sodium bicarbonate, sodium carbonate and sodium citrate.

The present formulation will generally comprise a pH adjuster in an amount in the range of about 0.01-10% by wt, if used in the formulation. The amount and type of pH adjuster will vary with the other components in the formulation. For example, the use of film forming polymers that are more basic in nature will require an acidic pH adjuster in an amount sufficient to yield a target pH of about 3.0 to about 7.5. Likewise, the use of film-forming polymers that are acidic in nature will require a basic pH adjuster in an amount sufficient to yield a target pH of about 3.0 to about 7.5. Less or more pH adjuster will be required based on the degree of acidity or basicity of the formulation.

The present formulation may also include a thickening agent to make the product more viscous. Examples of suitable thickening agents include, but are not limited to, acrylic acid polymer, sodium salt of acrylic acid polymer, acrylate polymer, polyurethane, xanthan gum, guar hydroxypropyltrimonium chloride, or a polysaccharide such as carrageenan.

The present formulation comprises at least one hair product ingredient including, but not limited to conditioning agents, nutrients, protectants, foaming agents, fragrances, colouring agents, preservatives and the like. The present formulation will generally comprise the hair product ingredient(s) in an amount in the range of 0.01-80% by wt.

Conditioning agents are used to improve the feel, texture, appearance and manageability of hair. Conditioning agents work to reduce friction between hair strands. There are different classes of conditioning agents, including cationic surfactants, humectants, emollients and oils, occlusives, proteins, silicones and polymers. Conditioning agents work by covering the hair strands, locking in moisture and reducing friction between strands. Examples of conditioning agents suitable for use in a hair care formulation include, but are not limited to, glycerin, propylene glycol, sorbitol, sodium salt of pyroglutamic acid, hyaluronic acid, panthenol, sodium lactate, coconut oil, argan oil, almond oil, olive oil, dimethicone, petrolatum, cyclomethicone, amodimethicone, polyquaternium-7, polyquaternium-11, benzalkonium chloride and mixtures thereof.

Nutrients aid in the overall health of the hair, including shine, thickness and strength. Examples of nutrients suitable for use in a hair care formulation include, but are not limited to, biotin, vitamins C and E, collagen, folic acid, omega-3 fatty acids and mixtures thereof.

A foaming agent is a surfactant, which in small amounts facilitates the formation of a foam, or enhances its colloidal stability by inhibiting the coalescence of bubbles. Surfactants, including foaming agents, are amphiphilic compounds that contain hydrophobic and hydrophilic groups. They reduce the surface tension between two liquids and the interfacial tension between a liquid and a solid. Examples of foaming agents suitable for use in a hair care formulation include, but are not limited to, disodium cocoyl glutamate, sodium cocoyl glutamate, sodium lauryl sulfoacetate and sodium cocoyl sulfate. Mixtures may also be used.

Hair protectants, such as heat protectants, protect hair when a heat-producing device, such as a blow dryer, straightener or curling iron, is used on hair. The extreme heat can cause damage to the pigments, keratin proteins, and cuticle, and reduce moisture inside the hair. Heat protectants are often composed of silicones or other polymers that work by decreasing the transfer of heat to the hair shaft reducing potential damage to hair. Heat protectants include, but are not limited to Quaternary 70, PVP/Dmapa acrylate copolymer, hydrolyzed wheat protein, dimethicone, cyclomethicone, polyquaternium, acrylate copolymer, cetearyl methicone, cetyl dimethicone, dimethiconol, stearyl dimethicone and mixtures thereof.

Fragrances provide a pleasant aroma to the products in which they are found, including hair care products. Fragrances suitable for use include natural and synthetic fragrance, or combinations thereof. Exemplary fragrances include, but are not limited to: limonene, sabinene, p-cymene, α-pinene, myrcene, β-elemene, germacrene D, β-caryophyllene, geraniol, farmesol, citronellol, methanol, nerol, linalool, terpinen-4-ol, bisabolol, carvacrol, thymol, geranyl acetate, linalyl acetate, neryl acetate, 1,8-cineole (eucalyptol), rose oxide, carvone, menthone, camphor, neral, citral, citronellal, anethole, myristicin, apiol and eugenol. Fragrances may also include perfumes and musks such as iso e Super®, galaxolide, tonalide, galaxolidone, phantolide, celestolide, crysolide, traseolide, muscone, civetone, exaltone, muscenone, cyclopentadecanolide, exaltolide, pentalide, habanolide, globalide, nirvanolide, ambrettolide, muscolide and essential oils or blends of essential oils. The fragrance may also be a mixture of any of the foregoing.

The formulation may also contain coloring agents or colorants. The color agents may include natural colors and dyes suitable for use in hair such as aluminum powder, annatto, bismuth citrate, bismuth oxychloride, bronze powder, caramel, carmine, β-carotene, chromium hydroxide green, chromium oxide greens, copper powder, disodium EDTA-copper, ferric ammonium ferrocyanide, ferric ferrocyanide, guaiazulene, guanine, henna, iron oxides, manganese violet, mica, potassium sodium copper chlorophyllin (chlorophyllin-copper complex), pyrophyllite, titanium dioxide, ultramarines, zinc oxide and D&C and FD&C straight colors and lakes including FD&C Blue No. 1, D&C Blue No. 4, D&C Brown No. 1, FD&C Green No. 3, D&C Green No. 5, D&C Green No. 6, D&C Orange No. 4, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10 and D&C Yellow No. 11. FD&C and D&C dyes and their corresponding chemical structures are described in the Kirk-Othmer Encyclopedia of Chemical Technology, Volume 5, pages 857-884. The amount of colorant included in the formulation will be an amount sufficient to achieve the desired coloring effect.

The present dry formulation may be provided as a powder to the consumer in bulk or in pre-measured format such as in sachets, blister packs, capsules, tablets and the like. Depending on the form of the dry formulation, further processing of the formulation may be required. The pre-measured forms may be single use or multiple use dosages.

In one embodiment, the dry formulation for use in a hair care product is provided in tablet form. To prepare such tablets, binding agents, diluents and/or lubricant may be added to the formulation to facilitate tabletting. As one of skill in the art will appreciate, appropriate binders, diluents and lubricant are non-toxic, physiologically inert, and physically and chemically stable by themselves as well as in combination with ingredients within the present formulation.

The tablets are made using well-established tabletting methodology. For example, selected ingredients are blended, and then may be subject to wet or dry granulation, following which they are compressed into tablets of a selected shape and size. As one of skill in the art will appreciate, the size will vary with the selected ingredients, but will generally be in a size in the range of about 5-100 grams or more; however, tablets outside of this range may also be made. The size of the tablet, and the amount of film-forming ingredient and other ingredients will dictate the amount of water to be combined with the tablet to form the final hair care product. For tablets in the range of 5-100 grams, an amount of water of about 250 ml may be applicable.

Tablet binders or binding agents are the substances which are added either dry, or in liquid form during wet granulation, to form granules or to promote cohesive compacts for directly compressed tablets. Binders assist with the flow and compressibility of the formulation. Examples of binding agents suitable for use in the present formulation include, but are not limited to:
(a) dry tablet binders, such as cellulose, polyvinyl pyrrolidone (PVP), hydroxy propyl methyl cellulose (HPMC), sodium carboxymethyl cellulose, polyethylene glycol (PEG), and methyl cellulose.
(b) solution tablet binders, such as starch-pregelatinized, cellulose, gelatin, cellulose derivatives, polyvinyl pyrrolidone, starch, sucrose, mannitol, polyethylene glycol, and liquid glucose.

Diluents act as fillers in pharmaceutical tablets to increase weight and improve content uniformity. Diluents provide improved tablet properties such as enhanced cohesion and/or flow. Examples of suitable diluents include, but are not limited to, one or more of starches, hydrolyzed starches, partially pregelatinized starches, anhydrous lactose, lactose monohydrate, and sugar alcohols such as sorbitol, xylitol and mannitol.

The present dry formulation will generally comprise the binding agent and/or diluent in an amount in the range of about 2-80% by wt.

Lubricants prevent adherence of granule/powder to punch die/faces and promote smooth ejection from the die after compaction during tablet formation. Lubricants can also be used when compression is not involved such as in powder blends for filling into capsules to prevent adherence of granule/powder to equipment surfaces and dosator mechanisms and for coating the surface of multi-particulate dosage forms to inhibit agglomeration of individual particles. Examples of suitable lubricants for inclusion in a hair care tablet include, but are not limited to, one or more of magnesium stearate, talcum, stearic acid, maize starch, colloidal silicone dioxide, boric acid, sodium chloride, sodium benzoate, polyethylene glycol 1000 and polyethylene glycol 6000.

The present formula will generally comprise the lubricant in an amount in the range of about 0.1% to about 10% by wt.

The formulation may also contain a disintegrant. Disintegrants are used to enhance the disintegration process of a tablet when contacted with fluid. Disintegrants, such as those that amplify the action of capillary forces and encourage the absorption of water, enlarge upon contact with water, or liberate gases leading directly to the disintegration of the tablet. Examples of disintegrants that may be added to the formulation include, for example, calcium carbonate, alginic acid, calcium carboxymethylcellulose, croscarmellose sodium, crospovidone, polacrilin potassium and sodium starch glycolate. Disintegrants may be incorporated in the formulation in amounts up to 40 wt %, e.g. 0-40% by wt.

The formulation may also contain a glidant. Glidants are blended with the formulation to enhance the tablet-core blend-material flow property. During the early stage of compression, glidants are mixed within the particle arrangement of the tablet powder blend to improve flowability and uniformity within the die cavity of tablet presses. Glidants encourage the flow of tablet granulation by diminishing friction between particles. Examples of glidants that may be added to the formulation are silicon dioxide, ascorbyl palmitate, magnesium stearate, starch and talc. Glidants may be incorporated in amounts up to about 5 wt %.

The formulation, whether powder or for tablet or other dosage forms, may also contain a preservative to prevent or minimize physical, chemical or biological change in the formulation. Examples of preservatives that are suitable for use in a hair care formulation include potassium sorbate, sodium benzoate, phenoxyethanol, Vitamin C, Vitamin E, grapefruit seed extract, BHA (butylatedhydroxyanisole), BHT (butylatedhydroxytoulene), methyl paraben, ethyl paraben, propyl paraben, butyl paraben, benzyl alcohol, phenol, meta cresol, chloro cresol, benzoic acid, sorbic acid, thiomersal, phenylmercuric nitrate, bronopol, propylene glycol, benzylkonium chloride and benzethonium chloride. Preservatives may be incorporated in amounts up to about 20% by wt.

In another aspect of the invention, a kit is provided. The kit comprises an amount of the present dry formulation useful to prepare a hair care product, and a container. The dry formulation is as above described comprising a water soluble film-forming polymer; ii) at least one hair product ingredient, and iii) optionally, a pH adjuster in an amount sufficient to establish a pH in the range of about 3.0 to about 7.5. The dry formulation may be pre-measured and provided in sachets, blister packs, or other pre-packaged forms. The dry formulation may alternatively be provided as a bulk powder, in tablet form or in another appropriate form suitable for dissolution in water. In the case of tablets or other dosage form, the dry formulation will generally also include additional ingredients as above described to aid in the formation of the tablet or other form.

The container provided with the kit is suitable to receive the dry formulation and water to form the hair care product. The container may be a bottle or other container with an opening to permit the ingredients to be added to the container, and the hair product to be removed therefrom. The container may also be a container specifically for application of a given hair product to hair, for example, a spray bottle, an aerosolizing container, a container that facilitates formation of a foaming hair product (e.g. foam-producing container), a tube for a thicker hair product such as a gel, and the like. In some embodiments, the kit optionally comprises instructions for making the hair product.

In another aspect, a kit is provided comprising the ingredients useful to make the dry formulation to form the hair care product. Thus, the kit comprises: i) a water soluble film-forming polymer; ii) at least one hair product ingredient; iii) optionally, a pH adjuster in an amount sufficient to establish a pH in the range of about 3.0 to about 7.5; as well as, iv) a container suitable for application of a hair care product. Provision of the ingredients of the dry formulation not previously admixed may be helpful to extend the shelf life thereof, particularly ingredients that are subject to oxidation or other degradation. This will also permit the provision of hair products that are not available in dry form, such as essential oils, perfumes, musks and nutrients. The kit may also include instructions for combining the ingredients, for example, indicating amounts of each to be combined.

In another aspect of the invention, a method of preparing a hair care product is provided, including, but not limited to, hair spray, mousse or hair gel. The method comprises the step of combining an amount of the dry formulation with a sufficient amount of water or aqueous solution to yield the hair care product. As noted above, the dry formulation may be provided in bulk or pre-measured, or as one or more tablets or other forms.

To achieve the desired product, the dry formulation is dissolved in an amount of water or aqueous solution. In one embodiment, an amount of the dry formulation is dissolved in about 10 mL to about 1000 mL of an aqueous solution, including water. For clarity, the aqueous solution may be water admixed with one or more water soluble ingredients for inclusion in the hair product. The precise amount of solution/water will be selected depending on the desired product. Products that have a lower viscosity, such as hair spray, may be dissolved in more solution/water than products that are more viscous, such as hair gel. The solution/water used may be distilled water, or tap water. While water at room temperature is generally sufficient to achieve the desired product, warm or hot water may be used with some ingredients to facilitate solubilisation of the formulation and preparation of the product.

The present invention advantageously provides a consumer prepared alternative to current hair care products that provides significant benefits as compared to hair care products that are currently available. Because the present hair care product is provided as a dry formulation, it significantly reduces the amount of packaging required, thereby reducing waste. Moreover, since the hair care product is produced by the consumer, the consumer may re-use a container once the container is emptied of product, thereby further reducing single use plastic container waste. This will in turn lead to reductions in shipping and fuel costs. Another advantage provided by the present invention in view of the reduced size of the consumer product, is more efficient storage of the product which is especially useful when travelling. Provision of a non-liquid product is also beneficial for travelling to avoid messy spills and also in view of restrictions with respect to liquid products when travelling by air.

Embodiments of the invention are described in the following specific examples which are not to be construed as limiting.

### Example 1—Hairspray Formulation

The following dry ingredients were combined to form a dry hair spray formulation:

| | | |
|---|---|---|
| Hydrolyzed corn starch (film former) | 30 | g |
| Citric acid (pH adjuster) | 0.5 | g |
| Potassium sorbate (preservative) | 1 | g |

The dry ingredients were then admixed with 250 mL of water to form a hair spray. Hair ingredients are optionally added such as fragrance, and/or hair nutrients/protectants to either the dry ingredients or to the water when combining.

### Example 2—Hair Mousse Formulation

The following dry ingredients are combined to form a dry hair mousse formulation:

| | | |
|---|---|---|
| Hydrolyzed corn starch | 22 | g |
| Sodium cocoyl glutamate | 5 | g |
| Citric acid | 0.1 | g |
| Phenoxyethanol | 1.0 | g |

The dry ingredients are then admixed with 250 mL of water to form a hair mousse.

### Example 3: Hair Gel Formulation

The following dry ingredients are combined to form a dry hair gel formulation:

| | | |
|---|---|---|
| Hydrolyzed corn starch | 10.8 | g |
| Xanthan gum | 2.1 | g |
| Glycerin | 8.0 | g |
| Phenoxyethanol | 0.7 | g |

The dry ingredients are then admixed with 180 mL of water to form a hair gel.

1. A dry formulation useful to prepare a hair care product comprising: i) a water soluble film-forming polymer; ii) at least one hair product ingredient, and iii) optionally, a pH adjuster in an amount sufficient to establish a pH in the range of about 3.0 to about 7.5.

2. The formulation of claim 1, wherein the water soluble film-forming polymer is selected from the group consisting of hydrolyzed corn starch, natural gum, polysaccharide, polyvinyl pyrrollidone polymer (PVP), carbopol, polyacrylamide, polyacrylic acid, acrylate copolymer, styrene acrylate copolymer, hydroxyester acrylate copolymer, PVP acrylate copolymer, polyvinylpyrrolidone/vinyl acetate copolymer (VP/VA copolymer), vinyl acetate polymer, vinyl acrylate copolymer, polyurethane, acrylic polyurethane hydride polymer, polyvinyl alcohol, hydroxypropylmethyl cellulose, hydroxyethyl cellulose, hydroxypropyl cellulose, carboxymethyl cellulose, glycolide, polylactide, methyl methacrylate copolymer, carboxyvinyl polymer, polyquaternium polymer, butyl or ethyl esters of polyvinyl methyl ether/maleic acid (PVM/MA) copolymers, and guar derivatives.

3. The formulation of claim 1, wherein the hair product ingredient is selected from conditioning agents, nutrients, protectants, foaming agents, fragrances, colouring agents and preservatives.

4. The formulation of claim 1, wherein the pH adjuster is selected from lactic acid, glycolic acid, citric acid, sodium bicarbonate, sodium carbonate and sodium citrate.

5. The formulation of claim 1, additionally comprising a thickening agent selected from acrylic acid polymer, sodium salt of acrylic acid polymer, acrylate polymer, polyurethane, xanthan gum, guar hydroxypropyltrimonium chloride and a polysaccharide.

6. The formulation of claim 1, comprising water soluble film forming polymers in an amount in the range of about 5% to about 99.9% by wt, hair product ingredient(s) in an amount in the range of 0.01-80% by wt, and optionally pH adjuster in an amount in the range of about 0.01-10% by wt.

7. The formulation of claim 1, provided in powder form.

8. The formulation of claim 1, provided in pre-measured forms.

9. The formulation of claim 1, provided in tablet form.

10. A kit comprising: i) an amount of a dry formulation useful to prepare a hair care product as defined in claim 1; and ii) a container.

11. A hair product tablet comprising a dry formulation as defined in claim 1 combined with a binding agent, diluent and/or a lubricant.

12. The hair product tablet of claim 11, comprising the binding agent and/or diluent in an amount in the range of about 2-80% by wt. and/or lubricant in an amount in the range of about 0.1% to about 10% by wt.

13. The hair product tablet of claim 11, additionally comprising a lubricant selected from magnesium stearate, talcum, stearic acid, maize starch, colloidal silicone dioxide, boric acid, sodium chloride, sodium benzoate, polyethylene glycol 1000 and polyethylene glycol 6000.

14. The hair product tablet of claim 11, additionally comprising a diluent selected from starches, hydrolyzed starches, partially pregelatinized starches, anhydrous lactose, lactose monohydrate, and sugar alcohols such as sorbitol, xylitol and mannitol.

15. A kit comprising:
i) one or more tablets as defined in claim 11; and
ii) a container.

16. A kit comprising:
i) one or more tablets comprising a water soluble film-forming polymer, and a binding agent, diluent and/or a lubricant; and
ii) a container suitable for application of a hair care product.

17. The kit of claim 16, wherein the container is a spray bottle, an aerosolizing container, a foam-producing container and a tube.

18. A kit comprising: i) a water soluble film-forming polymer; ii) at least one hair product ingredient; iii) optionally, a pH adjuster in an amount sufficient to establish a pH in the range of about 3.0 to about 7.5; and iv) a container suitable for application of a hair care product.

19. A method of making a hair care product comprising the step of: combining a formulation as defined in claim 1 or a tablet comprising the formulation, with a sufficient amount of an aqueous solution or water to yield the hair care product.
